(19) United States
(12) Patent Application Publication
(10) Pub. No.: US 2011/0224711 A1
(43) Pub. Date: Sep. 15, 2011

(54) Ring Handled Device and Method of Manufacturing Same

(76) Inventors: Jeffrey I. Lasner, Purchase, NY (US); Michael E. Lasner, Mt. Kisco, NY (US)

(21) Appl. No.: 13/042,246

(22) Filed: Mar. 7, 2011

Related U.S. Application Data
(60) Provisional application No. 61/339,797, filed on Mar. 9, 2010.

(51) Int. Cl. A61B 17/32; B26B 13/12; B21K 11/08; B21D 53/64

(52) U.S. Cl. 606/174; 30/254; 76/106.5

(57) ABSTRACT

A device, and a method of making the device, having at least one blank having a handle end and an operative end are described. The device may include a handle end having spaced complementary formations configured to co-unite to form a ring-shaped handle end. The method may include forming spaced complementary formations in the handle end of the blank, and co-uniting the spaced complementary formations to form a ring-shaped handle end in the blank.

### CROSS-REFERENCE TO RELATED APPLICATIONS

The present application claims the benefit of U.S. Provisional Patent Application No. 61/339,797, filed on Mar. 9, 2010, which is expressly incorporated herein in its entirety by reference thereto.

Additionally, U.S. Pat. Nos. 4,527,331; 6,592,603; and 7,497,867 are expressly incorporated herein in their entireties by reference thereto.

### FIELD OF THE INVENTION

The present invention relates to a ring handled device, and to a method of manufacturing the ring handled device.

### BACKGROUND INFORMATION

U.S. Pat. Nos. 4,527,331; 6,592,603; and 7,497,867 describe the use of materials, manufacture and cutting efficiencies for improved scissors or forceps devices over conventional self-opening or automatic return scissors or forceps devices.

### SUMMARY

Exemplary embodiments of the present invention provide a ring handled device, and a method of manufacturing the ring handled device.

According to an exemplary embodiment of the present invention, a device includes at least one blank having a handle end and an operative end, in which the handle end includes spaced complementary formations configured to co-unite to form a ring-shaped handle end.

The blank may be laser cut and/or stamped. The handle end may be configured to be twisted 90 degrees relative to the operative end, and the operative end may include a cutting blade and/or a grasping arm. In addition, the blank may include a pivot point situated between the handle end and the operative end. The spaced complementary formations may include a notch and a cutout, the cutout being situated between the notch and the pivot point. The device may include a first blank configured to be pivotally joined at the pivot point to a second blank, and the pivot point may include a rivet, a screw, and/or a pin.

The features of a method of manufacturing the ring handled device may have similar advantages as the features of the ring handled device.

According to an exemplary embodiment of the present invention, a device includes: a first component formed of a blank having a handle end and an operative end; and a second component formed of a blank having a handle end and an operative end, the first component and the second component pivotally coupled together. The handle end of the first component includes spaced complementary formations configured to co-unite to form a ring-shaped handle end, and the handle end of the second component includes spaced complementary formations configured to co-unite to form a ring-shaped handle end. The device thus forms a ring handle scissor and/or a ring handle blunt dissector.

According to an exemplary embodiment of the present invention, a method of making a device includes at least one blank having a handle end and an operative end, may comprise forming spaced complementary formations in the handle end of the at least one blank, and co-uniting the spaced complementary formations to form a ring-shaped handle end in the at least one blank.

The blank may be formed by laser cutting and/or stamping. The method may further include twisting the handle end 90 degrees relative to the operative end of the at least one blank. Further, the method may include forming a cutting blade and/or a grasping arm in the operative end of the at least one blank. In addition, the blank may include a pivot point situated between the handle end and the operative end. In the forming step, the spaced complementary formations may be formed as a notch and a cutout in the handle end of the at least one blank, the cutout being situated between the notch and the pivot point. The method may further include pivotally joining a first blank at the pivot point to a second blank, and the pivot point may include one of a rivet, a screw, and a pin.

Other features and aspects of example embodiments of the present invention are described in more detail below with reference to the appended Figures.

### BRIEF DESCRIPTION OF THE DRAWINGS

FIG. 1A illustrates a schematic plan view of a blank for a device according to an example embodiment of the present invention.

FIG. 1B illustrates a schematic side view of the blank.

FIG. 2 illustrates a schematic plan view of the blank, in which the handle end has been twisted 90 degrees relative to the operative end.

FIG. 3A illustrates a schematic plan view of the blank, in which the spaced complementary formations of the handle end have been co-united. FIG. 3B illustrates a schematic side view of the blank as shown in FIG. 3A, viewed in the direction indicated by arrow B.

FIG. 3C illustrates a schematic side view of the blank as shown in FIG. 3A, viewed in the direction indicated by arrow C.

FIG. 4 illustrates a schematic plan view of two blanks for a device according to an example embodiment the present invention.

FIG. 5 illustrates a schematic plan view of the two blanks, in which the two blanks have been pivotally joined at the pivot point.

FIG. 6 illustrates a schematic flow diagram of a method of making a device according to an example embodiment of the present invention.

### DETAILED DESCRIPTION

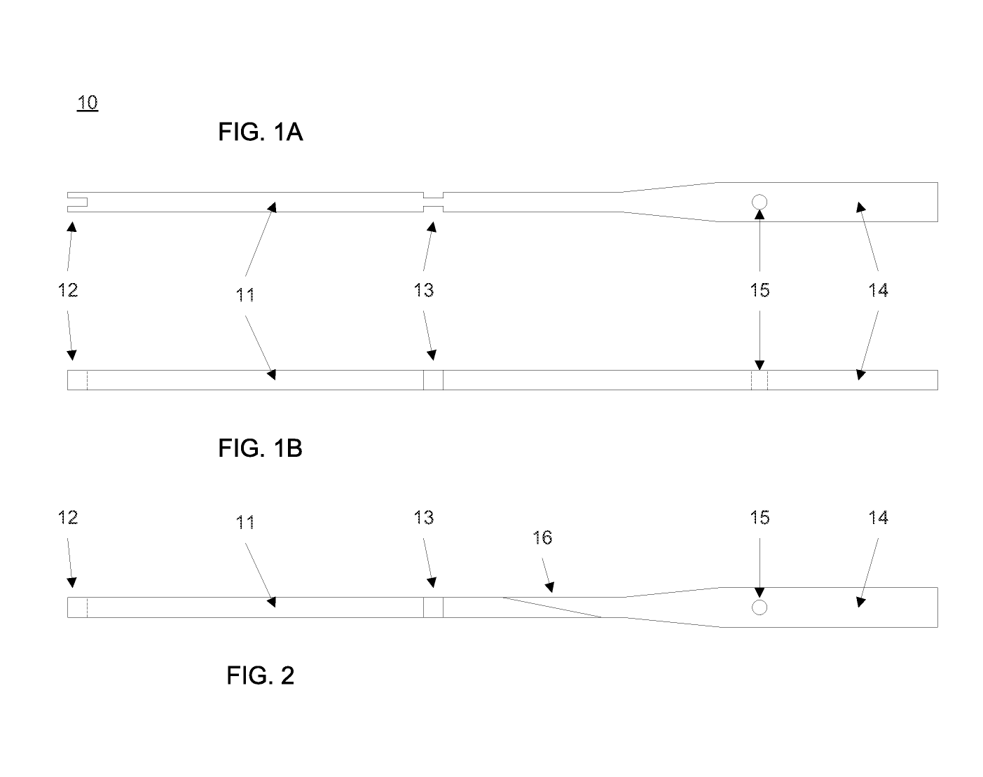

FIGS. 1A and 1B schematically illustrate a blank 10 for a device according to an exemplary embodiment of the present invention. FIG. 1A illustrates a plan view of the blank 10, and FIG. 1B illustrates a side view of the blank 10.

As shown in FIGS. 1A and 1B, the blank 10 may include a handle end 11 and an operative end 14. The blank 10 may also include a pivot point 15 situated between the handle end 11 and the operative end 14. The handle end 11 may include spaced complementary formations 12, 13. In this regard, a first formation 12 may be formed at a tip of the handle end 11, and a second formation 13 may be formed between the pivot point 15 and the first formation 12. For example, the spaced complementary formations 12, 13 may comprise a notch 12 formed at a tip of the handle end 11, and a cutout 13 formed between the pivot point 15 and the notch 12. Alternatively, other complementary formations 12, 13 may be used in place of the notch 12 and cutout 13 as shown in FIGS. 1A and 1B. For example, a projection and a hole may be used in place of the notch and cutout as the spaced complementary formations 12, 13. Furthermore, the spaced complementary formations 12, 13 may be formed in any shape and at various locations on handle end 11 such that the formations 12, 13 may be co-united as described herein. Although the complementary spaced formations 12, 13 may be referred to as a notch 12 and cutout 13 in the discussion herein, it is understood that the formations 12, 13 are not limited to the exemplary embodiment of a notch 12 and cutout 13.

The blank 10 may be formed by laser cutting and/or by stamping. For example, the handle end 11, notch 12, cutout 13, operative end 14, and pivot point 15 may be formed in one forming step by laser cutting or by stamping. Alternatively, other manufacturing methods may be used to produce the blank 10 including the handle end 11, notch 12, cutout 13, operative end 14, and pivot point 15.

FIG. 2 schematically illustrates the blank 10, in which the handle end 11 has been twisted 90 degrees relative to the operative end 14. The twist 16 may be situated between the handle end 11 and the operative end 14. More particularly, the twist 16 may be situated between the cutout 13 and the pivot point 15. Further, additional twists may be incorporated into the blank 10 at various points along its length, depending on the device to be formed. For example, a second twist (not shown) of 90 degrees may be situated between the operative end 14 and the pivot point 15 in order to form, for example, forceps or a hemostat or needle holder. Any known forming methods may be used to form the twist 16 between the handle end 11 and the operative end 14, as well as any additional twists.

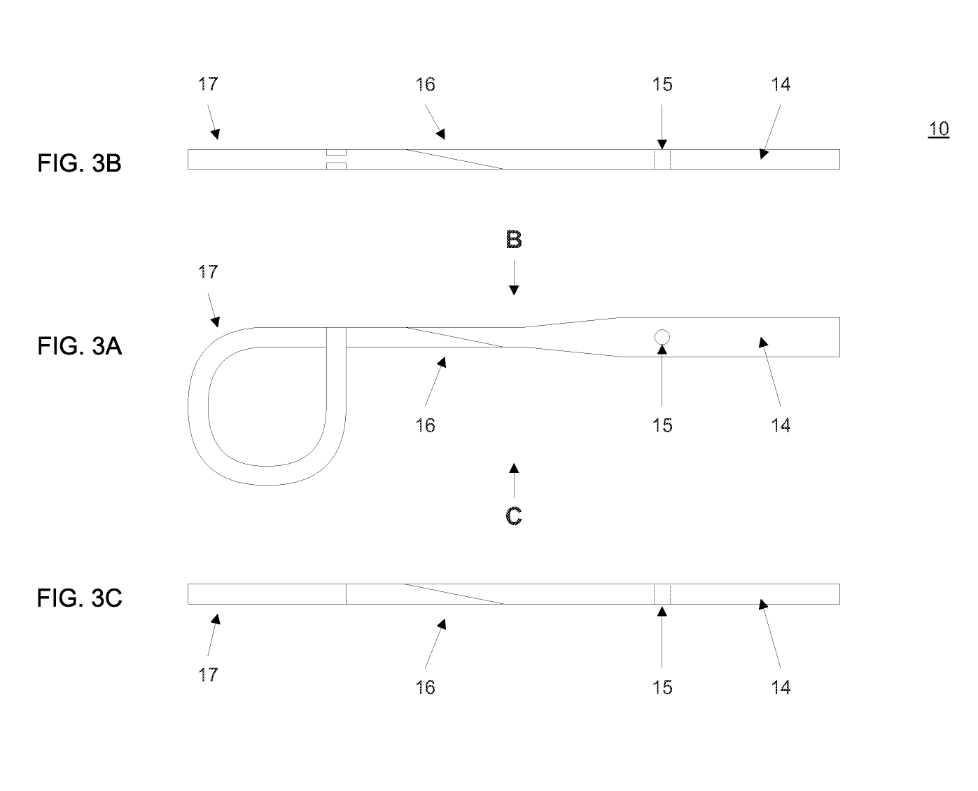

FIG. 3A illustrates a plan view of the blank 10, in which the spaced complementary formations 12, 13 of the handle end 11 have been co-united. FIG. 3B illustrates a side view of the blank 10 as shown in FIG. 3A, viewed in the direction indicated by arrow B. FIG. 3C illustrates a side view of the blank 10 as shown in FIG. 3A, viewed in the direction indicated by arrow C.

As shown in FIGS. 3A to 3C, the handle end 11 may be wrapped around in the form of an arc such that the notch 12 situated at the tip of the handle end 11 co-unites with the cutout 13 situated between the notch 12 and the twist 16. When the formations 12, 13 are co-united, a ring-shaped handle end 17 may be formed. Additionally, the formations 12, 13 may be welded after being co-united, in order to fix the ring-shaped handle end 17. Alternatively, other joining methods, such as adhesives, may be used to fix the ring-shaped handle end 17. However, depending on the particular arrangement of the spaced complementary formations 12, 13, such welding or other fixing methods may not be necessary to maintain the shape of the ring-shaped handle end 17.

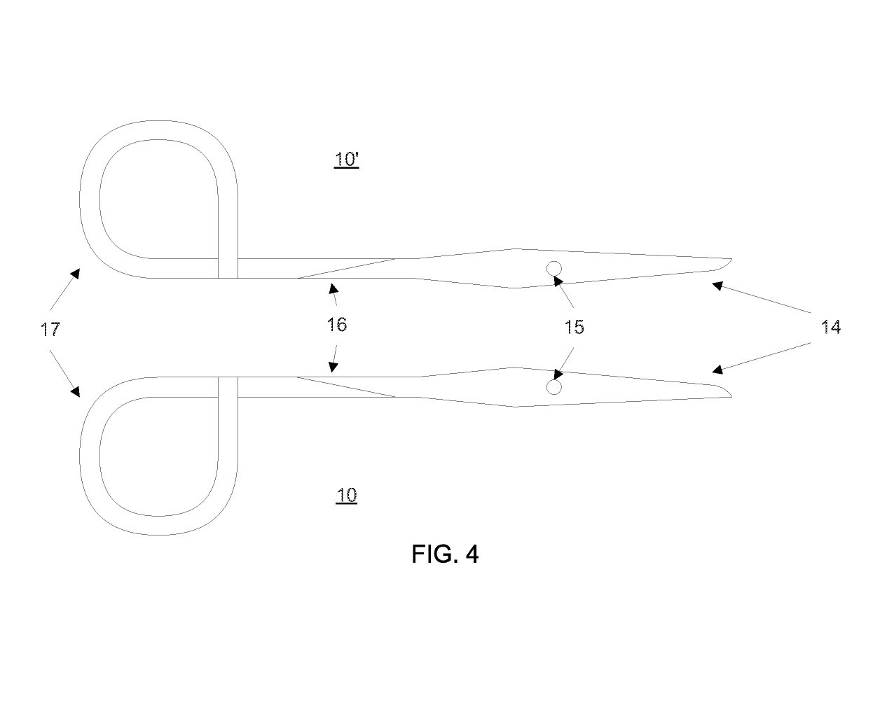

FIG. 4 schematically illustrates two blanks 10, 10′, may include analogous features including the ring-shaped handle end 17, twist 16, pivot point 15, and operative end 14 as described above. In addition, the operative end 14 of each blank 10, 10′ may be formed into an operative shape to function as scissors, forceps, blunt dissectors, spreaders, and/or other similar devices. For example, the operative ends 14 may be formed as cutting blades for scissors, grasping arms for forceps, and/or other configurations for similar devices. The two blanks 10, 10′ may be formed in similar manner, as described herein.

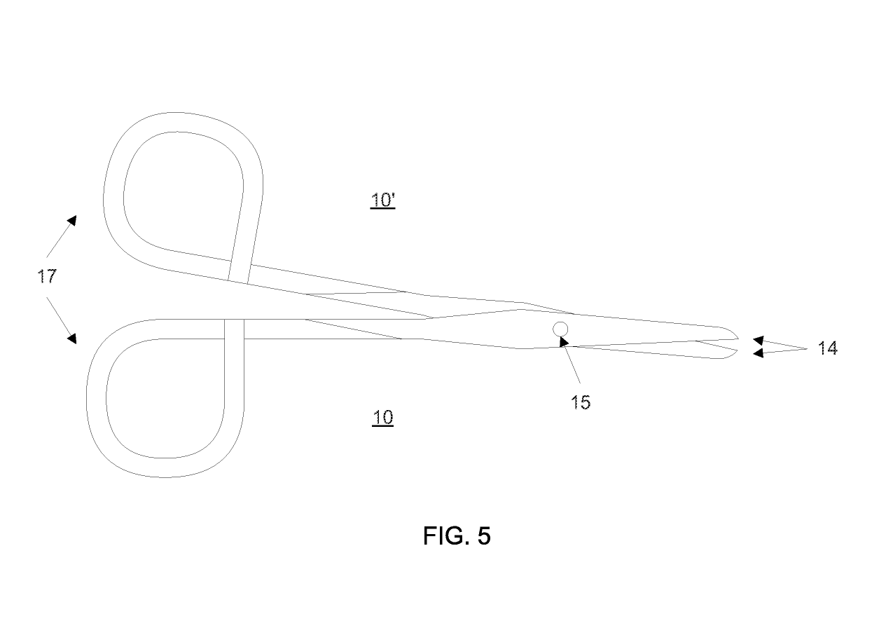

FIG. 5 schematically illustrates the two blanks 10, 10′, in which the two blanks 10, 10′ have been pivotally joined at the pivot point 15. The two blanks 10, 10′ may be joined at the pivot point 15 by a rivet, a screw, a pin, and/or any other method of pivotally joining the two blanks 10, 10′. FIG. 5 illustrates an assembled device in which the operative ends 14 are formed as a pair of scissors or blunt dissectors. Alternatively, the assembled device may include operative ends 14 formed as forceps and/or other similar devices.

The blank 10 may be made from flexibly resilient, medical grade materials having any or all the features described in U.S. Pat. Nos. 4,527,331; 6,592,603; and 7,497,867, which are expressly incorporated herein in their entireties by reference thereto.

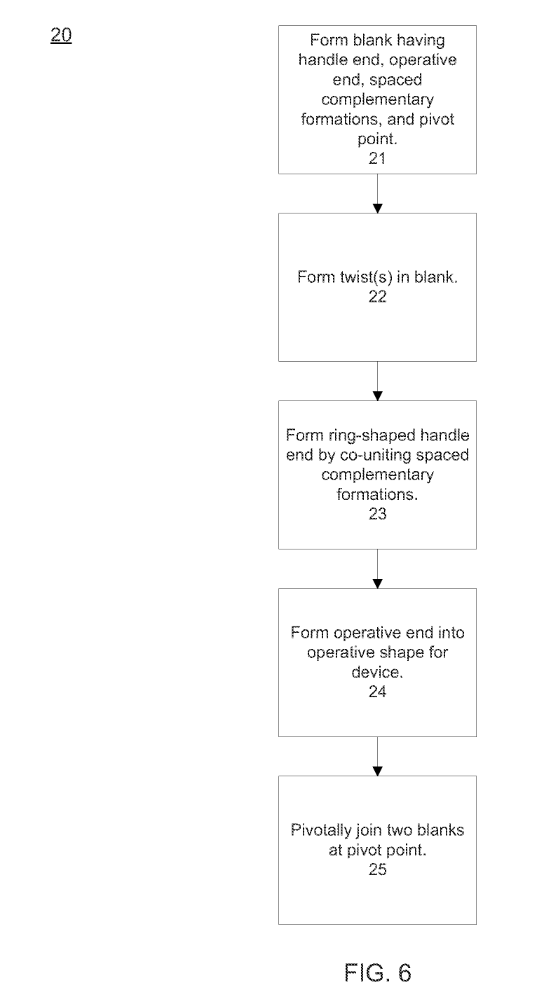

FIG. 6 illustrates a schematic flow diagram of a method 20 of making a device according to an example embodiment of the present invention.

At step 21, a blank 10 is formed having a handle end 11 and an operative end 14, such as an that described in U.S. Pat. Nos. 4,527,331; 6,592,603; and 7,497,867, which are expressly incorporated herein in their entireties by reference thereto. The blank 10 is also formed with a pivot point 15. Further, the handle end 11 of the blank 10 is formed with complementary spaced formations 12, 13. The blank 10 may be formed by laser cutting and/or by stamping. For example, the blank 10 may be formed in one forming step by laser cutting and/or by stamping. Alternatively, other manufacturing methods may be used to produce the blank 10.

At step 22, a twist 16 of, e.g., 90 degrees is formed in the blank 10. For example, the twist 16 may be situated between the handle end 11 and the operative end 14. More particularly, the twist 16 may be situated between the cutout 13 and the pivot point 15. Further, additional twists may be incorporated into the blank 10 at various points along its length, depending on the device to be formed, such as, for example, forceps, a hemostat, a needle holder, etc. In addition, although twists of 90 degrees have been mentioned, it is possible to form twists of various angles, also depending on the device to be formed. Any known forming methods may be used to form the twist(s) in the blank 10.

At step 23, a ring-shaped handle end 17 is formed by wrapping the handle end 11 around in the form of an arc such that the spaced complementary formations 12, 13 co-unite. Additional joining methods, such as welding or adhesives, may be used to fix the formations 12, 13 together.

At step 24, the operative end 14 of blank 10 is formed into an operative shape, for example, a cutting blade for scissors, a grasping arm for forceps, and/or other configurations for similar devices. Any known forming methods may be used to form the operative end 14 in the blank 10.

At step 25, a first blank 10 is pivotally joined at the pivot point 15 to a second blank 10′ in order to form a device, such as a pair of scissors, a blunt dissector, forceps and/or other similar devices. The two blanks 10, 10′ may be joined at the pivot point 15 by a rivet, a screw, a pin, and/or any other method of pivotally joining the two blanks 10, 10′.

Although the above steps 21 to 25 of the exemplary method 20 have been presented in a particular order, it should be understood that variations in the order of the steps 21 to 25 may be possible. For example, the order of steps 22, 23 and 24 for forming twist(s), forming a ring-shaped handle, and forming operative ends may be interchanged, and do not necessarily need to be performed in the order described above. Moreover, the operative end 14 of the blank 10 may be formed into its operative shape during step 21 of forming the handle end 11 and spaced complementary formations 12, 13. Further, other variations and/or combinations of the steps 21 to 25 of the method 20 may also be possible.

The method 20 may facilitate making ring handled scissors, blunt dissector, forceps and/or other devices more efficiently and with fewer steps than the conventional method of manufacturing ring handled devices.

In addition to the above described exemplary embodiments, other useful devices may also be manufactured according to the exemplary method of the present invention. Further, although the above described embodiments have been shown with a particular shape, size, and other features, the above embodiments are described by way of example only.

It is apparent that many modifications and variations of this invention as hereinabove set forth may be made without departing from the spirit and scope hereof. The specific embodiments described are given by way of example only, and the invention is limited only by the terms of the appended claims.

What is claimed is:

1. A device, comprising:
at least one blank having a handle end and an operative end;
wherein the handle end includes spaced complementary formations configured to co-unite to form a ring-shaped handle end.

2. The device according to claim 1, wherein the at least one blank is at least one of laser cut and stamped.

3. The device according to claim 1, wherein the handle end is configured to be twisted 90 degrees relative to the operative end.

4. The device according to claim 1, wherein the operative end includes at least one of one of a cutting blade, a blunt dissector, and a grasping arm.

5. The device according to claim 1, wherein the blank includes a pivot point situated between the handle end and the operative end.

6. The device according to claim 5, wherein the spaced complementary formations include a notch and a cutout, the cutout being situated between the notch and the pivot point.

7. The device according to claim 5, wherein a first blank is configured to be pivotally joined at the pivot point to a second blank of the at least one blank.

8. The device according to claim 5, wherein the pivot point includes at least one of a rivet, a screw, and a pin.

9. A device, comprising:
a first component formed of a blank having a handle end and an operative end;
a second component formed of a blank having a handle end and an operative end, the first component and the second component pivotally coupled together;
wherein the handle end of the first component includes spaced complementary formations configured to co-unite to form a ring-shaped handle end;
wherein the handle end of the second component includes spaced complementary formations configured to co-unite to form a ring-shaped handle end;
the device forming at least one of a ring handle scissor and a ring handle blunt dissector.

10. A method of making a device including at least one blank having a handle end and an operative end, comprising:
forming spaced complementary formations in the handle end of the blank; and
co-uniting the spaced complementary formations to form a ring-shaped handle end in the at least one blank.

11. The method according to claim 10, wherein the blank is formed by at least one of laser cutting and stamping.

12. The method according to claim 10, further comprising twisting the handle end 90 degrees relative to the operative end of the at least one blank.

13. The method according to claim 10, further comprising forming at least one of a cutting blade, a blunt dissector, and a grasping arm in the operative end of the blank.

14. The method according to claim 10, wherein a pivot point situated between the handle end and the operative end is formed in the blank.

15. The method according to claim 14, wherein in the forming step, the spaced complementary formations are formed as a notch and a cutout in the handle end of the blank, the cutout being situated between the notch and the pivot point.

16. The method according to claim 14, further comprising pivotally joining a first blank at the pivot point to a second blank.

17. The method according to claim 14, wherein the pivot point includes at least one of a rivet, a screw, and a pin.
